(19) United States
(12) Patent Application Publication
(10) Pub. No.: US 2026/0066117 A1
(43) Pub. Date: Mar. 5, 2026

(54) Artificial intelligence (AI) for survival prediction of cancer patients

(71) Applicant: REGENTS OF THE UNIVERSITY OF MICHIGAN, Ann Arbor, MI (US)

(72) Inventors: Lubomir M. Hadjiyski, Ann Arbor, MI (US); Vikas Gulani, Ann Arbor, MI (US); Di Sun, Ann Arbor, MI (US)

(21) Appl. No.: 19/305,108

(22) Filed: Aug. 20, 2025

Related U.S. Application Data
(60) Provisional application No. 63/689,110, filed on Aug. 30, 2024.

(51) Int. Cl. G16H 50/20; G06T 7/00; G06T 7/11; G16H 15/00; G16H 30/40; G16H 50/30

(52) U.S. Cl. CPC G16H 50/20; G06T 7/0016; G06T 7/11; G16H 15/00; G16H 30/40; G16H 50/30; G06T 2207/10072; G06T 2207/20081; G06T 2207/20084; G06T 2207/30096

(57) ABSTRACT

The present disclosure relates to predicting a survival rate of a patient with cancer following a treatment. In some embodiments, one or more processors: apply a large language model to a clinical report to extract a plurality of clinical features; acquire a pre-treatment image and a post-treatment image of the patient with cancer; apply a segmentation algorithm on an annotated volume of interest (VOI) of the pre-treatment image to obtain a first segmented VOI; apply the segmentation algorithm on the annotated VOI of the post-treatment image to obtain a second segmented VOI; determine a plurality of radiomics features and a plurality of deep learning features from the first segmented VOI and the second segmented VOI; and apply a machine learning model to the plurality of clinical features, the plurality of radiomics features, and the plurality of deep learning features to predict the survival rate of the patient with cancer.

### CROSS REFERENCE TO RELATED APPLICATIONS

This application claims the benefit of U.S. Provisional Application No. 63/689,110, entitled “Artificial intelligence (AI) for survival prediction of cancer patients” (filed Aug. 30, 2024), the entirety of which is incorporated by reference herein.

### FIELD OF THE DISCLOSURE

The present disclosure relates to predicting health conditions and, more particularly, to techniques for predicting a survival rate of a patient with cancer following a treatment.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Determining a survival rate of a patient with cancer following a treatment plays an important role in evaluating the effectiveness of the treatment and guiding future medical decisions. For example, survival rates provide insight into the likelihood of a patient surviving for a specified period after treatment, helping clinicians assess the potential benefits and risks of different therapeutic options. Despite their importance, accurately predicting survival rates remains challenging, and current techniques often produce an inaccurate survival rate prediction of a patient. A better method to determine the survival rate of a patient is needed.

### SUMMARY OF THE INVENTION

In one aspect, a computer-implemented method for predicting a survival rate of a patient with cancer following a treatment may be provided. The method may include: obtaining, by one or more processors, a clinical report of the patient with cancer; applying, by the one or more processors, a large language model to the clinical report to extract a plurality of clinical features; acquiring, by the one or more processors, a pre-treatment image and a post-treatment image of the patient with cancer, wherein a volume of interest (VOI) is annotated for each of the pre-treatment image and the post-treatment image; applying, by the one or more processors, a segmentation algorithm on the annotated VOI of the pre-treatment image to obtain a first segmented VOI; applying, by the one or more processors, the segmentation algorithm on the annotated VOI of the post-treatment image to obtain a second segmented VOI; determining, by the one or more processors, a plurality of radiomics features and a plurality of deep learning features from the first segmented VOI and the second segmented VOI; and/or applying, by the one or more processors, a machine learning model to the plurality of clinical features, the plurality of radiomics features, and the plurality of deep learning features to predict the survival rate of the patient with cancer. The method may include additional, fewer, or alternate actions, including those discussed else-where herein.

In another aspect, a computer system for predicting a survival rate of a patient with cancer following a treatment may be provided. The computer system may include: one or more processors; and/or one or more non-transitory memories coupled to the one or more processors. The one or more non-transitory memories may include computer-executable instructions stored therein that, when executed by the one or more processors, may cause the one or more processors to: system to: obtain a clinical report of the patient with cancer; apply a large language model to the clinical report to extract a plurality of clinical features; acquire a pre-treatment image and a post-treatment image of the patient with cancer, wherein a volume of interest (VOI) is annotated for each of the pre-treatment image and the post-treatment image; apply a segmentation algorithm on the annotated VOI of the pre-treatment image to obtain a first segmented VOI; apply the segmentation algorithm on the annotated VOI of the post-treatment image to obtain a second segmented VOI; determine a plurality of radiomics features and a plurality of deep learning features from the first segmented VOI and the second segmented VOI; and/or apply a machine learning model to the plurality of clinical features, the plurality of radiomics features, and the plurality of deep learning features to predict the survival rate of the patient with cancer. The computer system may include additional, less, or alternate functionality, including that discussed elsewhere herein.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic diagram illustrating one implementation of predicting a survival rate of a patient, in accordance with techniques described herein.

FIG. 2 is a block diagram of a survival prediction server, in accordance with various aspects of the present disclosure.

FIG. 3 depicts a flow diagram of predicting a survival rate of a patient following a treatment, in accordance with various aspects of the present disclosure.

FIG. 4 depicts images of volume of interest (VOI), in accordance with various aspects of the present disclosure.

FIG. 5 depicts images of region of interest (ROI), in accordance with various aspects of the present disclosure.

FIG. 6 depicts a flow diagram of a deep learning model, in accordance with various aspects of the present disclosure.

FIG. 7 depicts a neural network of a machine learning model, in accordance with various aspects of the present disclosure.

FIG. 8 depicts example charts of survival rate analysis, in accordance with various aspects of the present disclosure.

FIG. 9 depicts a flow diagram of an example method for predicting a survival rate of a patient, in accordance with various aspects of the present disclosure.

### DETAILED DESCRIPTION

Broadly speaking, the techniques of the present disclosure relate to an improved method of predicting a survival rate of a patient with cancer following a treatment. In some examples, one or more processors (e.g., of a survival prediction server) may be configured to: (i) obtain a clinical report of the patient with cancer; (ii) apply a large language model and/or a small language model to the clinical report to extract a plurality of clinical features (e.g., descriptors); (iii) acquire a pre-treatment image and a post treatment image of the patient with cancer; (iv) determine a plurality of radiomics features (e.g., descriptors) and a plurality of deep learning features (e.g., descriptors) from the pre-treatment image and the post-treatment image; and (v) apply a machine learning model to the plurality of clinical features, radiomics features, and deep learning features to predict the survival rate of the patent. In this manner, techniques of the present disclosure utilize a combination of clinical features, radiomics features, and deep learning features to predict the survival rate of the patient with cancer. As will be seen, these techniques improve over conventional techniques at least by utilizing the plurality of clinical features obtained from the large language model along with the radiomics features and the deep learning features in predicting the survival rate.

Conventional techniques do not, among other things, account for a combination of the clinical features, the radiomics features, and the deep learning features when predicting the survival rate of the patient with cancer. These conventional techniques therefore experience significant drawbacks, such as poor prediction of the survival rate of the patient with cancer.

By contrast, techniques of the present disclosure overcome these challenges of conventional techniques, and thereby provide multiple technical advantages over such conventional techniques. For instance, utilizing all three features improves the accuracy of the prediction of a survival rate of a patient with cancer.

Moreover, in some embodiments, the system utilizes both a pre-treatment image and a post-treatment image of the patient when determining with the plurality of radiomics features and the plurality of deep learning features. Comparing these images enables embodiments disclosed herein to more accurately assess the effectiveness of the treatment by identifying changes over time, leading to more precise radiomics and deep learning feature extraction. For example, utilizing only a post-treatment image to extract radiomics features may fail to capture baseline characteristics of the affected area (e.g., from the pre-treatment image), which may lead to incomplete or misleading conclusions about treatment efficacy.

The techniques of the present disclosure thus improve the functionality of a computing device (e.g., a hosting server such as a central server) at least by developing and using the clinical features, the radiomics features, and the deep learning features to predict the survival rate of the patient, and using both the pre-treatment image and the post-treatment image in determining both the radiomics features and the deep learning features. Techniques described herein improve the functioning of the computer itself at least because the computing device more effectively utilizes the data to extract features that help the computing device determine a more accurate prediction of the survival rate. This therefore improves over the prior art at least because existing systems do not determine the survival rate based on the combination of the clinical features, the radiomics features, and the deep learning features.

### Example System for Predicting a Survival Rate of a Patient with Cancer Following a Treatment

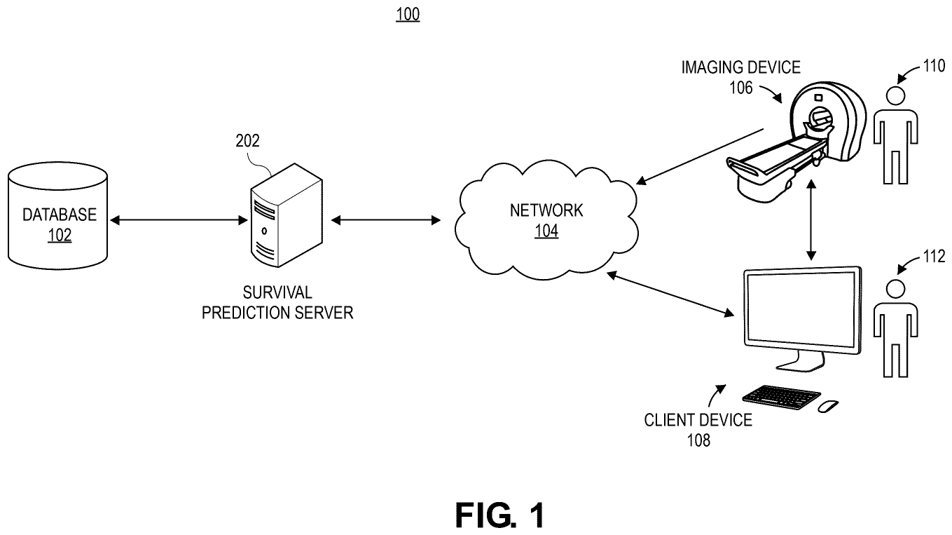

FIG. 1 depicts an example survival prediction system 100 including a survival prediction server 202, an imaging device 106, and a client device 108. The imaging device 106 and the client device 108 may be communicatively connected to each other. The survival prediction server 202 may be communicatively connected to the imaging device 106 and the client device 108 through a network 104. In an embodiment, the survival prediction server 202 may communicate via wireless signals over the network 104 with the imaging device 106 and the client device 108. The network 104 be any suitable local or wide area network(s) including a Wi-Fi network, a Bluetooth network, a cellular network such as 3G, 4G, Long-Term Evolution (LTE), 5G, the Internet, etc. In some instances, the imaging device 106 and the client device 108 may communicate with the digital network 104 via an intervening wireless or wired device, which may be a wireless router, a wireless repeater, a base transceiver station of a mobile telephony provider, etc.

The client device 108 may include, by way of example, a tablet computer, a network-enabled cell phone, a personal digital assistant (PDA), a mobile device, a smart-phone, a laptop computer, a desktop computer, a portable media player, a wearable computing device (e.g., smart glasses), a smart watch, a phablet, any device configured for wired or wireless RF (Radio Frequency) communication, etc. It should be appreciated that the client device 108 may include one or more processors, one or more memories, one or more display devices, etc. The client device 108 may be used by clinician 112.

The imaging device 106 may include, by way of example, a computed tomography (CT) imaging device, a magnetic resonance imaging (MRI) device, an ultrasound imaging device, a positron emission tomography (PET) imaging device, a single-photon emission computed tomography (SPECT) device, an X-ray fluoroscopy device, or any other suitable imaging system. Additionally, the imaging device may comprise hybrid imaging systems, such as PET-CT, PET-MRI, or SPECT-CT, to provide comprehensive diagnostic information.

The client device 108 may interact with the imaging device 106 to transmit a clinical report (e.g., an electronic clinical report, an electronic medical record (EMR), etc.), a pre-treatment image, and a post-treatment image of a patient 110 with cancer following a treatment and receive a survival rate of the patient 110. In some embodiments, the imaging device 106 may transmit the pre-treatment image and the post-treatment image directly. The client devices 108 may enable users to access the survival prediction server 202 from different environments and contexts. Upon receiving the clinical report, the pre-treatment image, and the post-treatment image, the survival prediction server 202 may process the input to determine a plurality of clinical features, deep learning features, and/or radiomics features. The survival prediction server 202 may then use the plurality of clinical features, deep learning features, and/or radiomics features to determine the survival rate of a patient 110 following the treatment. The survival prediction server 202 may then transmit the survival rate to the client device 108.

The survival prediction server 202 may be connected to a database 102. The database 102 may store information associated with a plurality of patients 110 including clinical data, pre-treatment images, post-treatment images, radiomics features, deep learning features, etc. The database 102 may also store training data that the survival prediction server 202 may use to train its large language model, feature extraction model, deep learning model, and prediction model. Furthermore, although the following description refers to a large language model, it should be appreciated that the techniques described herein may additionally or alternatively use a small language model and/or a hybrid language model (e.g., a model that blends any of a rule-based system, a statistical model, a machine learning approach, etc.).

It should be appreciated that any of the survival prediction server 202, the imaging device 106 and/or the client device 108 may include a display device configured to display any of the information discussed herein.

It should further be appreciated that although the example system 100 illustrates only one of each of the components, any number of the example components are contemplated (e.g., any number of databases, survival prediction servers, networks, imaging devices, patient, client devices, clinicians, etc.).

The large language model, the feature extraction model, the deep learning model, and the prediction model may be configured to implement machine learning, such that the model/engine “learns” to analyze, organize, and/or process data without being explicitly programmed. Machine learning may be implemented through machine learning methods and algorithms. In one exemplary embodiment, a machine learning module may be configured to implement machine learning methods and algorithms.

In some embodiments, at least one machine learning method and algorithm may be applied, which may include but is not limited to: linear or logistic regression, instance-based algorithms, regularization algorithms, decision trees, Bayesian networks, naïve Bayes algorithms, cluster analysis, association rule learning, neural networks (e.g., convolutional neural networks, deep learning neural networks, combined learning module or program, etc.), deep learning, combined learning, reinforcement learning, dimensionality reduction, support vector machines, k-nearest neighbor algorithms, random forest algorithms, gradient boosting algorithms, Bayesian program learning, voice recognition and synthesis algorithms, image or object recognition, optical character recognition, natural language understanding, and/or other machine learning programs/algorithms either individually or in combination. In various embodiments, the implemented machine learning methods and algorithms are directed toward at least one of several categorizations of machine learning, such as supervised learning, unsupervised learning, and reinforcement learning.

In one embodiment, the large language model, the feature extraction model, the deep learning model, and the prediction model may employ supervised learning, which involves identifying patterns in existing data to make predictions about subsequently received data. Specifically, the large language model, the feature extraction model, the deep learning model, and the prediction model may be “trained” using training data, which includes example inputs and associated example outputs. Based upon the training data, the large language model, the feature extraction model, the deep learning model, and the prediction model may generate a predictive function which maps outputs to inputs and may utilize the predictive function to generate machine learning outputs based upon data inputs.

In another embodiment, the large language model, the feature extraction model, the deep learning model, and the prediction model may employ unsupervised learning, which involves finding meaningful relationships in unorganized data. Unlike supervised learning, unsupervised learning does not involve user-initiated training based upon example inputs with associated outputs. Rather, in unsupervised learning, the large language model, the feature extraction model, the deep learning model, and the prediction model may organize unlabeled data according to a relationship determined by at least one machine learning method/algorithm employed by the similarity score model and M/S ratio model. Unorganized data may include any combination of data inputs and/or machine learning outputs as described above.

In yet another embodiment, the large language model, the feature extraction model, the deep learning model, and the prediction model may employ reinforcement learning, which involves optimizing outputs based upon feedback from a reward signal. Specifically, the large language model, the feature extraction model, the deep learning model, and the prediction model may receive a user-defined reward signal definition, receive a data input, utilize a decision-making model to generate a machine learning output based upon the data input, receive a reward signal based upon the reward signal definition and the machine learning output, and alter the decision-making model so as to receive a stronger reward signal for subsequently generated machine learning outputs. Other types of machine learning may also be employed, including deep or combined learning techniques.

After training, machine learning programs (or information generated by such machine learning programs) may be used to evaluate additional data. Such data may be and/or may be related to intent data, user device data, and/or other data that was not included in the training dataset. The trained machine learning programs (or programs utilizing models, parameters, or other data produced through the training process) may accordingly be used for determining, assessing, analyzing, predicting, estimating, evaluating, or otherwise processing new data not included in the training dataset.

It is to be understood that supervised machine learning and/or unsupervised machine learning may also comprise retraining, relearning, or otherwise updating models with new, or different, information, which may include information received, ingested, generated, or otherwise used over time.

Moreover, although the methods described elsewhere herein may not directly mention machine learning techniques, such methods may be read to include such machine learning for any determination or processing of data that may be accomplished using such techniques. In some aspects, such machine learning techniques may be implemented automatically upon occurrence of certain events or upon certain conditions being met. In any event, use of machine learning techniques, as described herein, may begin with training a machine learning program, or such techniques may begin with a previously trained machine learning program.

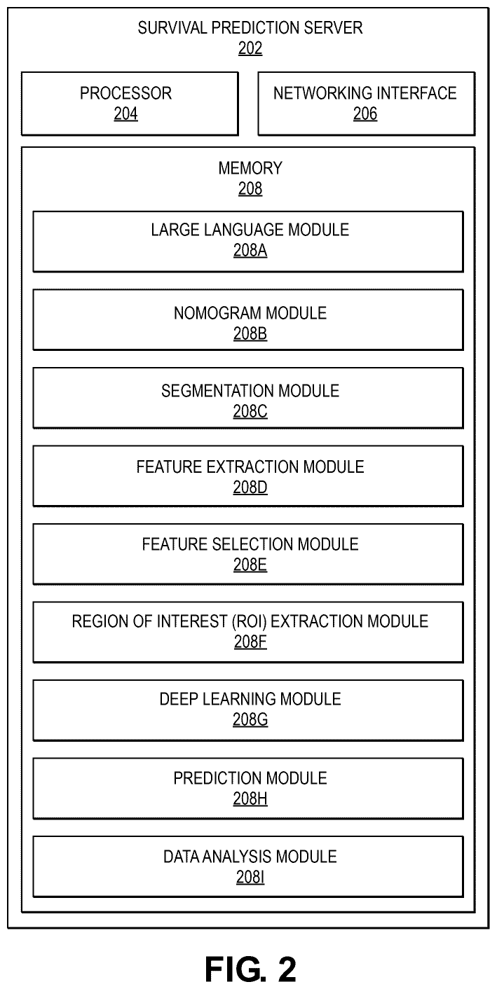

FIG. 2 is an expanded block diagram of a survival prediction server 202, in accordance with various aspects of the present disclosure. Generally speaking, the survival prediction server 202 may obtain a clinical report of a patient with cancer, apply a large language model to the clinical report to extract a plurality of clinical features, acquire a pre-treatment image and a post-treatment image of the patient with cancer, determine a plurality of radiomics features and a plurality of deep learning features from the images, and predict a survival rate of the patient with cancer using the clinical features, radiomics features, and the deep learning features. The survival prediction server 202 includes one or more processors 204, one or more memories 208, and a networking interface 206. The memories 206 include a large language module 208A, a nomogram module 208B, a segmentation module 208C, a feature extraction module 208D, a feature selection module 208E, a region of interest (ROI) extraction module 208F, a deep learning module 208G, a prediction module 208H, and a data analysis module 208I. In some embodiments, the survival prediction server 202 may be the survival prediction server 202 of FIG. 2.

The survival prediction server 202 may obtain a clinical report of a patient with cancer through the networking interface 206. The survival prediction server 202 may use a large language module 208A to determine a plurality of clinical features from the clinical report. The large language module 208A may comprise a large language model for extracting clinical features from the clinical report. The clinical features may be determined by the large language model using the clinical report and the prompt (e.g., asking for specific features to be extracted from the clinical report) given to the large language model. For example, the clinical features extracted using the large language model may include post-surgery pathologic stage, lymphovascular invasion (LVI), pathologic node stage, whether patients underwent neoadjuvant chemotherapy, whether patients underwent adjuvant radiotherapy, etc.

The large language model may be trained using a set of clinical reports and a set of clinical features extracted from the set of clinical reports through the supervised learning as described above. During the training, the model may learn to associate specific language patterns and terminologies with the corresponding clinical features. This process may enable the model to accurately identify and extract these features from new, unseen clinical reports. By continuously refining its understanding through exposure to diverse clinical data, the large language model can generalize across various types of reports, improving its ability to recognize complex medical information. As a result, when the survival prediction server 202 processes a clinical report, the large language model can efficiently extract relevant features, even if they are expressed in different formats or with subtle variations in language.

The survival prediction server 202 may then use the nomogram module 208B to predict a survival rate of a patient using the plurality of clinical features extracted from the large language module 208A. The nomogram module 208B may comprise a nomogram. The nomogram may convert each feature of the plurality of clinical features into a numerical value by mapping them to a point axis. Each feature may contribute to a certain number of points, thereby determining a plurality of points from the nomogram model. Upon determining the plurality of points, the points may be summed to calculate the total point for the patient. The nomogram module 208B may then map the total point to a survival probability axis to predict the survival rate of a patient.

The point axis and the survival probability axis may be predefined algorithms or machine learning models that may be derived from using large dataset and rigorous analyses. These axes may be developed through statistical methods such as Cox proportional hazards regression, where the relative importance of each clinical feature is quantified. The resulting coefficients may then be used to assign points to each feature based on their contribution to the survival outcome. The total points may then be mapped to a survival probability axis, which may be derived from the survival function estimated from the dataset.

As an illustrative example of using the nomogram, consider a patient diagnosed with a bladder cancer, and a clinician using a nomogram to estimate the patient's 5-year survival probability. The extracted data for the clinical features for the nomogram may be, for example: 1) post-surgery pathologic stage: stage II, 2) lymphovascular invasion (LVI): present, 3) pathologic node stage: N1 (1-3 positive nodes), 4) neoadjuvant chemotherapy: Yes, and 5) adjuvant radiotherapy: Yes. Each of the feature may then be converted into points using the point axis, with result being 1) stage II: 50 points, 2) LVI (Present): 40 points, 3) pathologic node stage N1: 30 points, 4) neoadjuvant chemotherapy (Yes): 20 points, and 5) adjuvant radiotherapy (No): 10 points. The sum of all these features may then be determined to be 150 points. The 150 points may then be mapped to the survival probability axis on the nomogram, which may correspond to a 5-year survival probability of 65%.

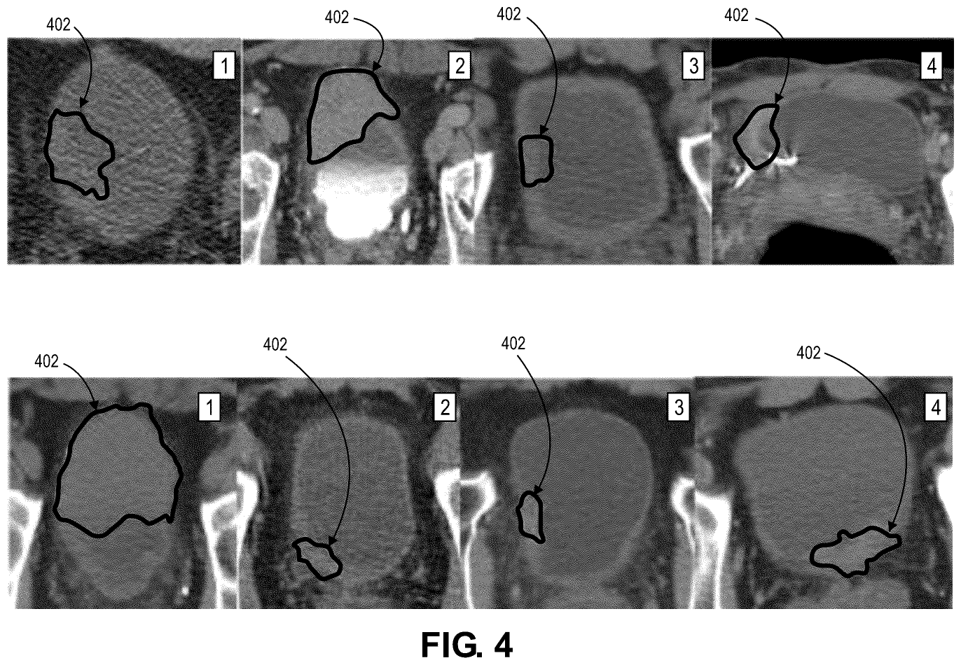

The survival prediction server 202 may acquire a pre-treatment image and a post-treatment image of the patient with cancer through the networking interface 206. The survival prediction server 202 may use a segmentation module 208C to segment a volume of interest (VOI) from the pre-treatment image and the post-treatment image to determine a first segmented VOI and a second segmented VOI. For example, the volume of interest may be a lesion area such as the tumor region, a specific organ, or a particular anatomical structure affected by the cancer. The segmentation process isolates this area from surrounding tissues, allowing for precise analysis and comparison between the pre-treatment and post-treatment images. The segmentation module 208C may comprise a segmentation model (algorithm) to segment the VOI. The segmentation model may be a machine learning model trained on labeled data to accurately identify and delineate the boundaries of the VOI within the images. In some embodiments, the VOI in the pre-treatment image and the post-treatment image may be highlighted by a physician, and the survival prediction server 202 may use the segmentation module 208C to segment the highlighted VOI. Example VOIs 402 are illustrated by FIG. 4.

The survival prediction server 202 may use the feature extraction module 208D to determine radiomics features. The radiomics features help reveal intricate tumoral patterns and characteristics that may not be perceivable by the naked eye. The feature extraction module 208D may comprise a feature extraction model to determine a set of radiomics features from the segmented pre-treatment image and the post-treatment image. The feature extraction model may be a machine learning model trained to extract meaningful features, such as radiomics features, such as morphological features (e.g., volume, circularity, rectangularity, Fourier descriptor, etc.), texture features (e.g., run length statistics, etc.), gradient field (e.g., the gradient magnitudes statistics for all voxels on the surface of the segmented VOI, etc.), and intensity based features (e.g., the average gray level and contrast features, etc.), etc. The feature extraction module 208D may use the feature extraction model to determine a first set of radiomics features for the pre-treatment image and a second set of radiomics features for the post-treatment image.

The feature extraction module 208D may additionally obtain a third set of radiomics features for changes in radiomics features between the first set of radiomics features and the second set of radiomics features. The third set of radiomics features may be obtained as follows:

$${f}_{diff} = \frac{{f}_{pre} - {f}_{post}}{{f}_{pre}}$$

where fₚᵣₑ is a feature in the first set of radiomics features, fₚₒₛₜ is a feature in the second set of radiomics features, and f_{diff} is a feature in the third set of radiomics feature.

In some embodiments, the total number of radiomics features in the first set, the second set, and the third set may be too many. For example, if there are 81 radiomics features, then there will be total of 273 radiomics features extracted from the first, second, and the third set. Therefore, the survival prediction server 202 may employ the feature selection module 208E to reduce the number of radiomics features. The feature selection module 208E may eliminate redundant features, reduce the time and space requirements for data processing, and mitigate the risk of the “curse of dimensionality” by focusing on the most relevant features. The feature selection module 208E may comprise mutual information method, which evaluates the relevance of features by measuring the information shared between each feature and the target variable (e.g., survival rate). The mutual information method assesses the dependency between features and the target outcome, identifying those features with the highest informational value. The survival prediction server 202 may therefore determine a plurality of radiomics features that are most relevant to predicting the survival rate.

In some other embodiments, the segmented VOI of the pre-treatment image and the post-treatment image may be too large for the survival prediction server 202 to extract deep learning features as inputs. Therefore, the survival prediction server 202 may use the region of interest (ROI) extraction module 208F to extract ROI images from the segmented VOI of the pre-treatment and the post-treatment image. The ROI module may use a sliding window technique to systematically scan the segmented VOI and extract smaller, focused regions that contain relevant features. The sliding window technique may involve moving a fixed-size window across the VOI and capturing overlapping or non-overlapping segments. By concentrating on these smaller, targeted regions, the ROI images help the deep learning model learn and analyze features more efficiently, reducing computational complexity and enhancing the model's ability to detect and interpret critical patterns within the data.

The ROI extraction module 208F may extract a first plurality of ROI images for the first segmented VOI and a second plurality of ROI images for the second segmented VOI. The ROI extraction module 208F may then combine each ROI image for the first plurality of ROI images with a matching ROI image of the second plurality of ROI images to determine a third plurality of ROI images. Each ROI image of the third plurality of ROI images may be a hybrid image that contains both the ROI image of the pre-treatment and the post-treatment. In some embodiments, the third plurality of ROI images may have a threshold limit to the number of the hybrid images to prevent biases towards larger segmented VOIs (e.g., image pairs including larger lesions). In some other embodiments, the ROI extraction module 208F may apply an enlargement technique (e.g., cubic spline interpolation) to each ROI image of the third plurality of ROI images to align with an input specification for the deep learning model.

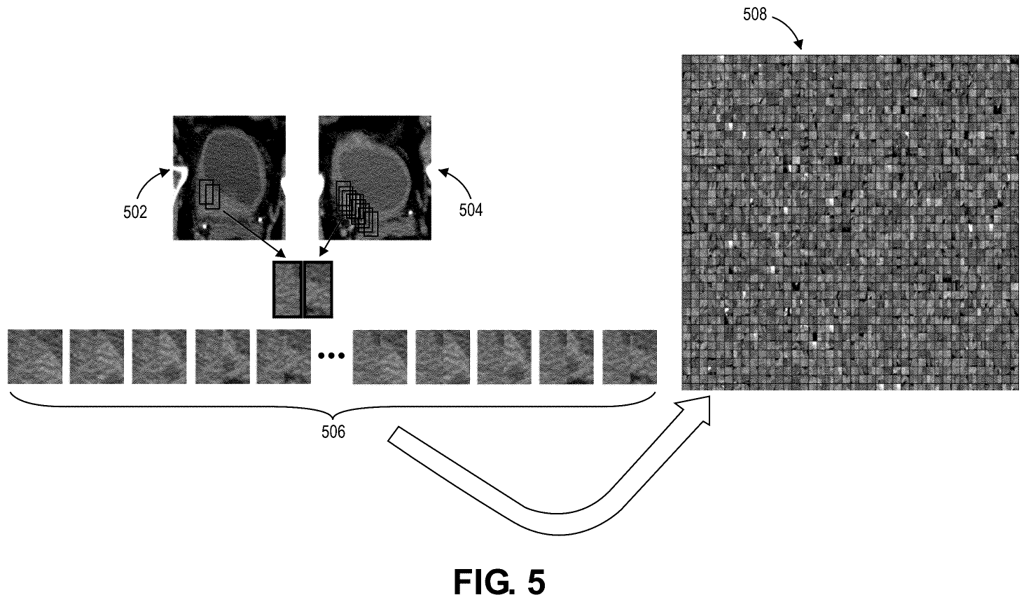

Examples of the hybrid image and the third plurality of ROI images are depicted in the example of FIG. 5. That is, the example of FIG. 5 illustrates pre-treatment image 502, post-treatment image 504, hybrid images 506, and subset of hybrid ROIs shown in matrix form 508.

The survival prediction server 202 may then proceed to determine a plurality of deep learning factors using the deep learning module 208G. The deep learning module 208G may have a deep learning model that is trained with labeled hybrid ROI image data. For example, the hybrid ROI image data for a patient that survived after 5 years may be marked with 1 while the hybrid ROI image data for a patient that did not survive after 5 years may be marked with 0. The deep learning model may then use this labeled data to train on patterns and features indicative of a survival rate of a patient. During training, the model learns to identify and differentiate between the complex features and patterns associated with each outcome. Once trained, the model can apply these learned patterns to new ROI images to predict the survival probability of future patients.

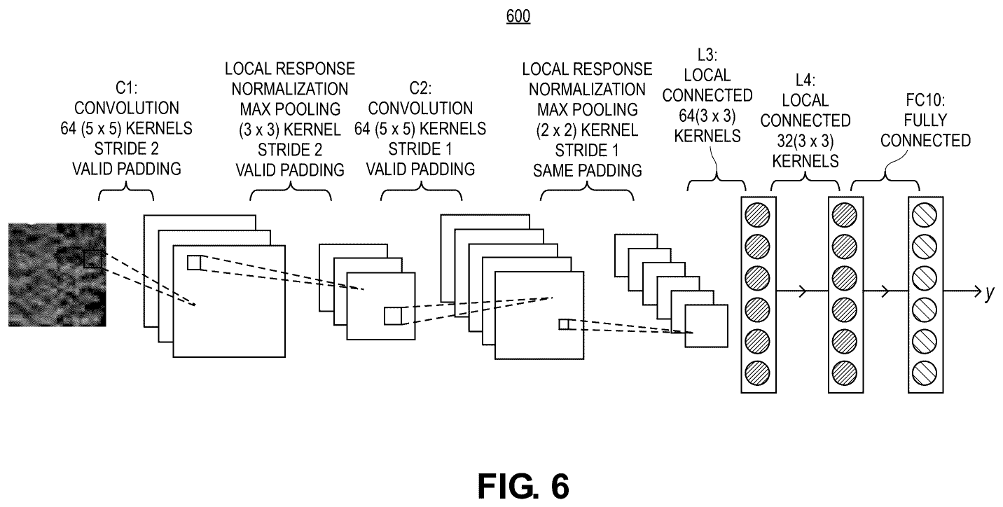

The deep learning model may comprise a convolutional neural network (CNN), which may be used to determine deep learning features, and a set of connected layers. In some embodiments, such as in the example implementation 600 of FIG. 6, the CNN may comprise two convolutional layers, C1 and C2, each of which may be accompanied by a local response normalization layer and a max-pooling layer. The CNN may determine the plurality of deep learning factors using the third plurality of ROI images. The set of connected layers may comprise two locally connected layers L3 and L4, and a fully connected layer FC10. The set of connected layers may determine the survival rate using the plurality of deep learning factors determined from the convolutional neural network. In this regard, FIG. 6 illustrates details for an example deep learning model in accordance with the techniques described herein.

In some examples, a first section of a deep learning neural network (e.g., the first four convolution/pooling layers) may be applied to the hybrid ROI images (e.g., the third plurality of ROI images described above with reference to FIG. 5) to determine the plurality of deep learning features. Additionally or alternatively, second section of the deep learning neural network (e.g., the last three fully connected layers) may be applied to the plurality of deep learning features to determine a survival likelihood score. Additionally or alternatively, the machine learning model may be applied to the plurality of clinical features, the plurality of radiomics features, and/or the survival likelihood score to predict the survival rate of the patient with cancer.

The survival prediction server 202 may use the prediction module 208H to determine the survival rate of a patient with cancer following a treatment. The prediction module 208H may use the plurality of clinical features, the plurality of radiomics features, and/or the plurality of deep learning features to determine a survival rate of a patient with cancer following a treatment. The prediction module 208H may comprise a prediction model, which may be a machine learning algorithm trained to integrate and analyze these diverse features. By leveraging patterns and correlations learned from historical data, the prediction model can combine clinical, radiomic, and deep learning features to predict the survival rate of a patient with cancer following a treatment.

The prediction module 208H may determine the survival rate based on different combinations of the plurality of clinical features, the plurality of radiomics features, and the plurality of deep learning features. For instance, the prediction model may determine the survival rate based on only radiomics features. In another example, the prediction model may determine the survival rate based on the clinical features and the deep learning features. Depending on the availably of different data, the prediction module 208H may be flexible with its use in determining the survival rate of a patient.

In some embodiments, the prediction module 208H may determine the survival rate using the points for the plurality of clinical features determined from using the nomogram module 208B instead of the plurality of clinical features. In some other embodiments, the prediction module 208H may use the survival rate determined from the plurality of deep learning factors instead of the plurality of deep learning factors to determine the survival rate of the patient.

Upon determining the survival rate of a patient using the nomogram module 208B, the deep learning module 208G, or the prediction module 208H, the survival prediction server 202 may use the data analysis module 208I to analyze each prediction. The data analysis module 208I may display different statistical relevance and highlight which combinations of the clinical features, radiomics features, and deep learning features are most accurate in generating predictions. This analysis may involve evaluating the performance of each feature set through metrics such as accuracy, precision, recall, AUC (Area Under the Curve), etc. Additionally, the module may identify key features that have the highest impact on prediction accuracy and assess how well different feature combinations contribute to the overall prediction model. By providing insights into the effectiveness of various features and their interactions, the data analysis module 208I helps refine the predictive models, optimize their performance, and guide clinical decision-making by identifying the most reliable indicators of patient survival.

### Example Technique for Predicting a Survival Rate of a Patient with Cancer Following a Treatment

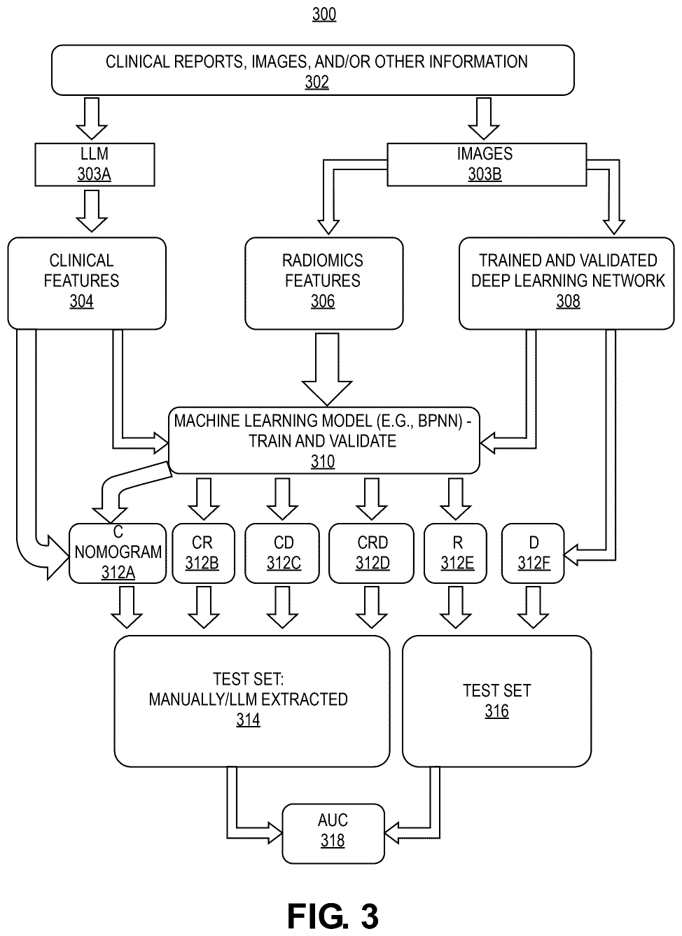

FIG. 3 depicts an example flow diagram 300 of predicting a survival rate of a patient following a treatment, in accordance with various aspects of the present disclosure. In some embodiments, the example flow diagram 300 may be implemented, wholly or partially, by any of the survival prediction server 202, the client device 108, and/or the imaging device 106. It should be appreciated that, as used herein, “C” represents clinical features; “R” represents radiomics features; “D” represents deep-learning features; “CR” represents clinical and radiomics features; “CD” represents clinical and deep-learning features; and “CRD” represents clinical, radiomics and deep-learning features.

In the example flow diagram 300, the clinical reports, images and/or other information 302 may be held by or received from (e.g., received by the one or more processors 204) the database 102. Large language model (LLM) 303A may be applied to the images to extract a plurality of clinical features 304. Furthermore, although the example of FIG. 3 illustrates LLM 303A, it should be appreciated that 303A may additionally or alternatively comprise a small language model and/or a hybrid language model (e.g., a model that blends any of a rule-based system, a statistical model, a machine learning approach, etc.). The clinical features 304 may be sent to the machine learning model 310, such as a back propagation neural network (BPNN), and such as for training and/or validation of the machine learning model 310.

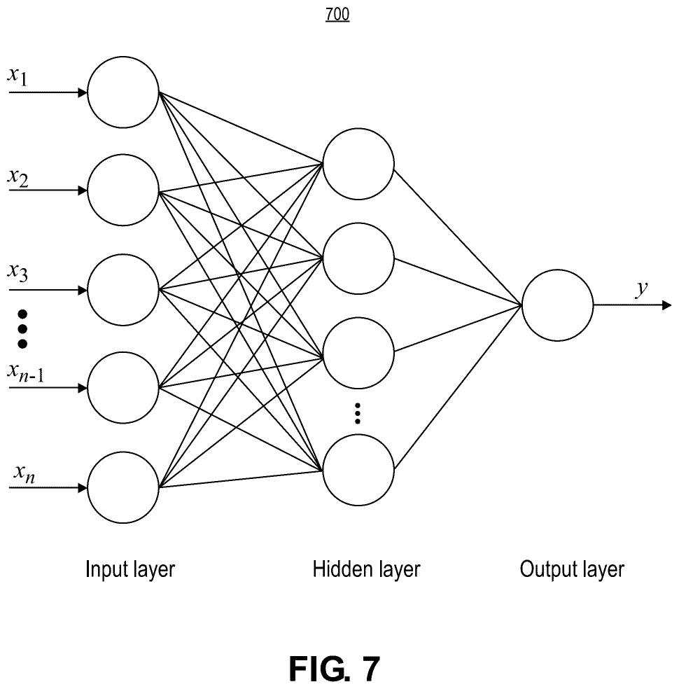

In some embodiments, the machine learning model 310 comprises a BPNN. An example BPNN 700 is depicted by FIG. 7. In the illustrated example, the input xᵢ=(1, 2, 3, . . . , n) are the features. The hidden layer contains thirteen nodes. The output y is the likelihood score assessing the survival of each patient.

The images 303B may also be sent to the deep learning network 308 (e.g., a deep learning convolutional neural network, etc.), such as to extract deep learning features 312F. In this regard, FIG. 6 depicts one example implementation 600 of the deep learning network 308.

Additionally or alternatively, radiomics features 306 may be determined from the images 303B. For example, as described above, feature extraction module 208D may determine the radiomics features 306.

The machine learning model may determine any or all of: clinical features 312A; clinical and radiomics features 312B; clinical and deep-learning features 312C; clinical, radiomics and deep-learning features 312D; and/or radiomics features 312E.

Any or all of the: clinical features 312A; clinical and radiomics features 312B; clinical and deep-learning features 312C; clinical, radiomics and deep-learning features 312D; radiomics features 312E; and/or deep learning features 312F may the be used to create the test set(s) 314, 316. In some embodiments, the test set 316 includes only automatically extracted features (e.g., features extracted by the machine learning model 310, the deep learning network 308, the LLM 303A, etc.); whereas, the test set 314 includes one or both of manually and/or automatically extracted features. One or both of the test set(s) 314, 316 may then be used to calculate the AUC 318.

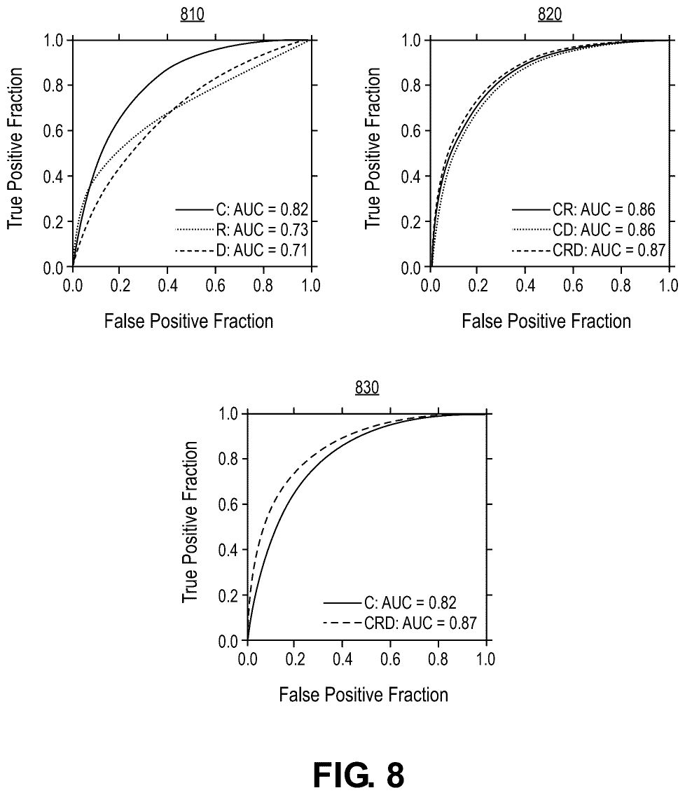

Example AUCs are illustrated by FIG. 8, which depicts example charts of survival rate analysis, in accordance with various aspects of the present disclosure. More specifically, example chart 810 depicts receiver operating characteristic (ROC) curves and area under curve (AUC) values for the individual feature. Example chart 820 depicts ROC curves and AUC values for combined features. Example chart 830 depicts a direct comparison of the ROC curves of the clinical features (C) to the clinical, radiomics, and deep learning (CRD) features.

### Example Method for Predicting a Survival Rate of a Patient with Cancer Following a Treatment

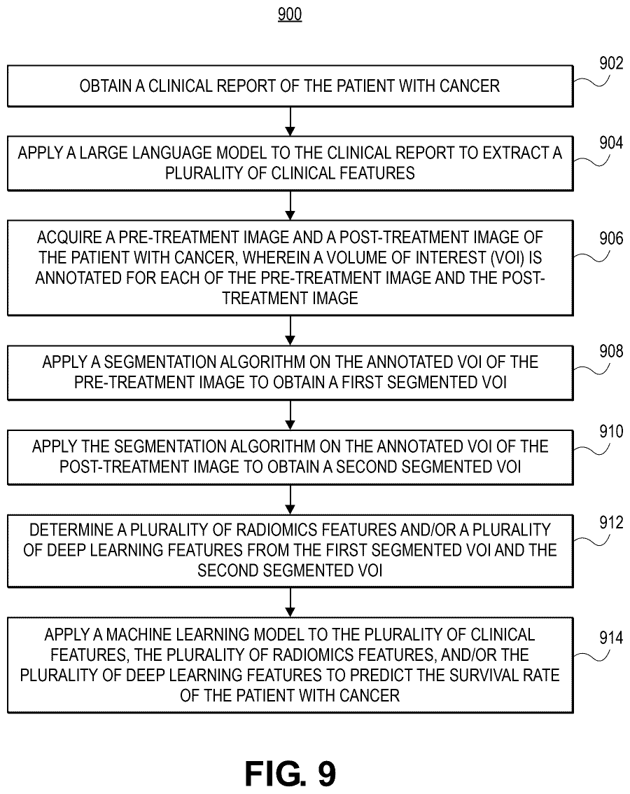

FIG. 9 depicts a flow diagram of an example method 900 for predicting a survival rate of a patient, in accordance with various aspects of the present disclosure. In some embodiments, the example method 900 may be implemented, wholly or partially, by any of the survival prediction server 202, the client device 108, and/or the imaging device 106.

The example method 900 may begin at block 902 when a clinical report is obtained. A large language model may be applied at block 904 to extract a plurality of clinical features. At block 906, a pre-treatment image and/or a post-treatment image of the patient with cancer may be acquired; and/or a volume of interest (VOI) may be annotated for one or both of the pre-treatment image and the post-treatment image.

At block 908, a segmentation algorithm may be applied on the annotated VOI of the pre-treatment image to obtain a first segmented VOI. At block 910, the same or different segmentation algorithm may be applied on the annotated VOI of the post-treatment image to obtain a second segmented VOI.

At block 912, a plurality of radiomics features and a plurality of deep learning features may be determined from the first segmented VOI and the second segmented VOI.

At block 914, a machine learning model may be applied to the plurality of clinical features, the plurality of radiomics features, and/or the plurality of deep learning features to predict the survival rate of the patient with cancer.

It should be understood that not all blocks and/or events of the exemplary diagrams and/or flowcharts are required to be performed. Moreover, the exemplary diagrams and/or flowcharts are not mutually exclusive (e.g., block(s)/events from each example diagram and/or flowchart may be performed in any other diagram and/or flowchart). The exemplary diagrams and/or flowcharts may include additional, less, or alternate functionality, including that discussed elsewhere herein.

### Other Matters

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A computer-implemented method for predicting a survival rate of a patient with cancer following a treatment, the method comprising:
obtaining, by one or more processors, a clinical report of the patient with cancer;
applying, by the one or more processors, a large language model to the clinical report to extract a plurality of clinical features;
acquiring, by the one or more processors, a pre-treatment image and a post-treatment image of the patient with cancer, wherein a volume of interest (VOI) is annotated for each of the pre-treatment image and the post-treatment image;
applying, by the one or more processors, a segmentation algorithm on the annotated VOI of the pre-treatment image to obtain a first segmented VOI;
applying, by the one or more processors, the segmentation algorithm on the annotated VOI of the post-treatment image to obtain a second segmented VOI;
determining, by the one or more processors, a plurality of radiomics features and a plurality of deep learning features from the first segmented VOI and the second segmented VOI; and
applying, by the one or more processors, a machine learning model to the plurality of clinical features, the plurality of radiomics features, and the plurality of deep learning features to predict the survival rate of the patient with cancer.

2. The computer-implemented method of claim 1, further comprising:
building, by the one or more processors, a nomogram model based on the plurality of clinical features;
determining, by the one or more processors, a plurality of points from the nomogram model; and
applying, by the one or more processors, the machine learning model to the plurality of points, the plurality of radiomics features, and/or the plurality of deep learning features to predict the survival rate of the patient with cancer.

3. The computer-implemented method of claim 1, wherein the determining the plurality of radiomics features includes:
applying, by the one or more processors, a feature extraction model on the first segmented VOI to determine a first plurality of radiomics features for the pre-treatment image; and
applying, by the one or more processors, the feature extraction model on the second segmented VOI to determine a second plurality of radiomics features for the post-treatment image.

4. The computer-implemented method of claim 3, further comprising:
determining, by the one or more processors, a third plurality of radiomics features for changes in features between the first plurality of radiomics features and the second plurality of radiomics features; and
applying, by the one or more processors, a feature selection algorithm on the first plurality of radiomics features, the second plurality of radiomics features, and the third plurality of radiomics features to determine the plurality of radiomics features.

5. The computer-implemented method of claim 4, wherein the feature selection algorithm is a mutual information method.

6. The computer-implemented method of claim 1, wherein the determining the plurality of deep learning features includes:
extracting, by the one or more processors, a first plurality of region of interest (ROI) images for the first segmented VOI;
extracting, by the one or more processors, a second plurality of ROI images for the second segmented VOI; and
combining, by the one or more processors, ROI images of the first plurality of ROI images with a corresponding ROI images of the second plurality of ROI images to determine a third plurality of ROI images comprising hybrid ROI images.

7. The computer-implemented method of claim 6, wherein the third plurality of ROI images have a threshold limit to a size of the third plurality of ROI images.

8. The computer-implemented method of claim 6, further comprising:
applying, by the one or more processors, a first section of a deep learning neural network to the third plurality of ROI images to determine the plurality of deep learning features.

9. The computer-implemented method of claim 8, further comprising:
applying, by the one or more processors, a second section of the deep learning neural network to the plurality of deep learning features to determine a survival likelihood score; and
applying, by the one or more processors, the machine learning model to the plurality of clinical features, the plurality of radiomics features, and/or the survival likelihood score to predict the survival rate of the patient with cancer.

10. The computer-implemented method of claim 6, further comprising:
applying, by the one or more processors, an enlargement technique to each ROI image of the third plurality of ROI images to align with an input specification for a deep learning neural network.

11. The computer-implemented method of claim 1, further comprising:
extracting, by the one or more processors, the plurality of clinical features by inputting, into the large language model: (i) the clinical report, and (ii) a prompt.

12. The computer-implemented method of claim 1, wherein the pre-treatment image or the post-treatment image is a computed tomography (CT) image, magnetic resonance imaging (MRI) image, ultrasound image, X-ray image, positron emission tomography (PET) image, and/or single photon emission computed tomography (SPET).

13. The computer-implemented method of claim 1, wherein the VOI is a lesion area.

14. A computer system for predicting a survival rate of a patient with cancer following a treatment, comprising:
one or more processors, and
a non-transitory program memory coupled to the one or more processors and storing executable instructions that, when executed by the one or more processors, cause the computer system to:
obtain a clinical report of the patient with cancer;
apply a large language model to the clinical report to extract a plurality of clinical features;
acquire a pre-treatment image and a post-treatment image of the patient with cancer, wherein a volume of interest (VOI) is annotated for each of the pre-treatment image and the post-treatment image;
apply a segmentation algorithm on the annotated VOI of the pre-treatment image to obtain a first segmented VOI;
apply the segmentation algorithm on the annotated VOI of the post-treatment image to obtain a second segmented VOI;
determine a plurality of radiomics features and a plurality of deep learning features from the first segmented VOI and the second segmented VOI; and
apply a machine learning model to the plurality of clinical features, the plurality of radiomics features, and the plurality of deep learning features to predict the survival rate of the patient with cancer.

15. The computer system of claim 14, wherein the instructions, when executed by the one or more processors, further cause the computer system to:
build a nomogram model based on the plurality of clinical features;
determine a plurality of points from the nomogram model; and
apply the machine learning model to the plurality of points, the plurality of radiomics features, and/or the plurality of deep learning features to predict the survival rate of the patient with cancer.

16. The computer system of claim 14, wherein the instructions, when executed by the one or more processors, further cause the computer system to:
apply a feature extraction model on the first segmented VOI to determine a first plurality of radiomics features for the pre-treatment image; and
apply the feature extraction model on the second segmented VOI to determine a second plurality of radiomics features for the post-treatment image.

17. The computer system of claim 16, wherein the instructions, when executed by the one or more processors, further cause the computer system to:
determine a third plurality of radiomics features for changes in features between the first plurality of radiomics features and the second plurality of radiomics features; and
apply a feature selection algorithm on the first plurality of radiomics features, the second plurality of radiomics features, and the third plurality of radiomics features to determine the plurality of radiomics features.

18. The computer system of claim 14, wherein the instructions, when executed by the one or more processors, further cause the computer system to:
extract a first plurality of region of interest (ROI) images for the first segmented VOI;
extract a second plurality of ROI images for the second segmented VOI; and
combine each ROI image of the first plurality of ROI images with a matching ROI image of the second plurality of ROI images to determine a third plurality of ROI images.

19. The computer system of claim 18, wherein the instructions, when executed by the one or more processors, further cause the computer system to:
apply a first section of a deep learning neural network to the third plurality of ROI images to determine the plurality of deep learning features.

20. The computer system of claim 19, wherein the instructions, when executed by the one or more processors, further cause the computer system to:
apply a second section of the deep learning neural network to the plurality of deep learning features to determine a survival likelihood score; and
apply the machine learning model to the plurality of clinical features, the plurality of radiomics features, and/or the survival likelihood score to predict the survival rate of the patient with cancer.
